(19) United States
(12) Patent Application Publication
(10) Pub. No.: US 2025/0090073 A1
(43) Pub. Date: Mar. 20, 2025

(54) FLEXIBLE AND SCALABLE MICROELECTRODE PROBE DEVICE

(71) Applicant: The Regents of the University of California, Oakland, CA (US)

(72) Inventors: Shadi A. Dayeh, San Diego, CA (US); Yun Goo Ro, Seoul (KR); Keundong Lee, San Diego, CA (US)

(21) Appl. No.: 18/882,087

(22) Filed: Sep. 11, 2024

Related U.S. Application Data
(60) Provisional application No. 63/584,578, filed on Sep. 22, 2023.
(60) Provisional application No. 63/582,653, filed on Sep. 14, 2023.

(51) Int. Cl. A61B 5/293; A61B 5/00; A61N 1/05

(52) U.S. Cl. CPC A61B 5/293; A61B 5/6846; A61N 1/0534; A61B 2560/063; A61B 2562/043; A61B 2562/227

(57) ABSTRACT

A microelectrode probe device includes multiple layers of biocompatible polymer films formed in an elongate shape. Micro-electrode recording conductive sites and macro-electrode conductive stimulation sites are exposed from the polymer films near a first terminal end of the elongate shape. Insulated electrical traces within the polymer films are connected to the micro-electrode recording conductive sites and macro-electrode conductive stimulation sites. An elongate hollow within the multiple layers of polymer films extends to micro-electrode recording conductive sites and macro-electrode conductive stimulation sites. The elongated hollow being configured to accommodate a removable stainless-steel stylet. The stainless-steel stylet permits implantation with the probe device wrapped around it, and the stylet can be removed.

### PRIORITY CLAIM AND REFERENCE TO RELATED APPLICATION

The application claims priority under 35 U.S.C. § 119 and all applicable statutes and treaties from prior U.S. provisional application Ser. No. 63/582,653, which was filed Sep. 14, 2023 and from prior U.S. provisional application Ser. No. 63/584,578, which was filed Sep. 22, 2023.

### STATEMENT OF GOVERNMENT INTEREST

This invention was made with government support under grant numbers UG3NS123723-01, R01NS123655-01, DP2-EB029757 awarded by the National Institutes of Health and from grant numbers 1728497, 1743694 and 1351980 awarded by the National Science Foundation. The government has certain rights in this invention.

### FIELD

A field of the invention is brain sensing and stimulation. The invention provides a neuro stimulation and monitoring array that can be delivered via a minimally invasive delivery device.

### BACKGROUND

Over the past few decades, a wide variety of neural probes have been fabricated and reported using Si ([1-3]) or metals ([4,5]). Such rigid materials risk damage of the soft brain tissue during implantation and suffer from signal degradation overtime due to chronic tissue inflammation. Biocompatible polymers made of softer materials such as paryleneC([6,7]), polymide ([8,9]) and SU-8([10]) have been considered as alternative materials and used, primarily in animal studies, as neural probes for long-term in vivo recordings. Implantation causes less microdamage to the surrounding brain tissue but deformation of flexible probes during implantation inhibits the ability to reach deep brain structures.

A rigid insertion shuttle can facilitate precise insertion of a flexible probe into the cortex ([11-15]). Insertion shuttles are attached to flexible polymer probes with bio-dissolvable adhesive and later extracted by dissolving adhesive after implantation ([11-14]. The insertion shuttles limit probe size and implantation location options primarily to superficial brain layers.

Hollow structure neural probes have been previously used for metal wire insertion using XeF₂ etching of Si ([15]). Typical prior probes have a constricted device length less than 10 mm. These devices can record/stimulate from superficial layers of the brain, mostly in animals, but are not suitable for deep brain recording and stimulation in humans and non-human primates.

One prior probe uses platinum-iridium microwires extruding from the tip of depth electrodes enable recording of single and multi-unit activity from up to 9 microwires. [16] This configuration only allows recording from the tip. Dixi Medical has produced a depth electrode with extensible microwires from the body of the array known as the MICRODEEP® multi-contact flexi-rigid intracerebral electrode, with a diameter of 0.8 mm. Neither of these approaches allows more than a few channels to be recorded, neither afford grid-like high spatial resolution in that developing a spatial map of multiple action potential sites of origin are not possible, and the devices are still hand-made.

Other electrodes that can record single units from the human brain and afford high resolution spatial mapping of single cell activity include the Utah array [17] and Neuropixels [18] with up to hundreds of channels [19], [20]. These devices, currently used in research, are limited by the silicon (Si) manufacturing technology and the brittleness of Si. They are also currently only able to access superficial cortical layers of the brain.

To increase the spatial resolution and channel count of electrodes that can record from either the lateral grey matter or deep brain structures, recent engineering approaches have focused on rolling or adhering conformable and photolithographically defined polyimide electrodes around or on medical-grade tubing used in clinical depth electrodes. [21]-[24]. These hybrid integration approaches impose a limitation on the size of the electrode such that the starting diameter is pre-determined by the clinical depth electrode diameter

### REFERENCE LIST

1 Campbell, P. K., Jones, K. E., Huber, R. J., Horch, K. W., & Normann, R. A. (1991). A silicon-based, three-dimensional neural interface: manufacturing processes for an intracortical electrode array. IEEE Transactions on Biomedical Engineering, 38(8), 758-768.
2 Vetter, R. J., Williams, J. C., Hetke, J. F., Nunamaker, E. A., & Kipke, D. R. (2004). Chronic neural recording using silicon-substrate microelectrode arrays implanted in cerebral cortex. IEEE transactions on biomedical engineering, 51(6), 896-904.
3 Blanche, T. J., Spacek, M. A., Hetke, J. F., & Swindale, N. V. (2005). Polytrodes: high-density silicon electrode arrays for large-scale multiunit recording. Journal of neurophysiology, 93(5), 2987-3000.
4 Loeb, G. E., Peck, R. A., & Martyniuk, J. (1995). Toward the ultimate metal microelectrode. Journal of neuroscience methods, 63(1-2), 175-183.
5 Schmidt, E. M., Bak, M. J., Hambrecht, F. T., Kufta, C. V., O'rourke, D. K., & Vallabhanath, P. (1996). Feasibility of a visual prosthesis for the blind based on intracortical micro stimulation of the visual cortex. Brain, 119(2), 507-522.
6 Kato, Y., Nishino, M., Saito, I., Suzuki, T., & Mabuchi, K. (2006). Flexible intracortical neural probe with biodegradable polymer for delivering bioactive components. In Microtechnologies in Medicine and Biology, 2006 International Conference on (pp. 143-146). IEEE.
7 Castagnola, V., Descamps, E., Lecestre, A., Dahan, L., Remaud, J., Nowak, L. G., & Bergaud, C. (2015). Parylene based flexible neural probes with PEDOT coated surface for brain stimulation and recording. Biosensors and Bioelectronics, 67, 450-457.
8 Rousche, P. J., Pellinen, D. S., Pivin, D. P., Williams, J. C., Vetter, R. J., & Kipke, D. R. (2001). Flexible polyimide based intracortical electrode arrays with bioactive capability. IEEE Transactions on biomedical engineering, 48(3), 361371.
9 Xiang, Z., Yen, S. C., Xue, N., Sun, T., Tsang, W. M., Zhang, S., Liao, L. D., Thakor, N. V. & Lee, C. (2014). Ultra-thin flexible polyimide neural probe embedded in a dissolvable maltose-coated microneedle. Journal of Micromechanics and Microengineering, 24(6), 065015
10 Altuna, A., de la Prida, L. M., Bellistri, E., Gabriel, G., Guimeri, A., Berganzo, J., Villa, R & Fernindez, L. J. (2012). SU-8 based microprobes with integrated planar electrodes for enhanced neural depth recording. Biosensors and Bioelectronics, 37(1), 1-5.
11 Kozai, T. D. Y., & Kipke, D. R. (2009). Insertion shuttle with carboxyl terminated self-assembled monolayer coatings for implanting flexible polymer neural probes in the brain. Journal of neuroscience methods, 184(2), 199-205.
12. Kuo, J. T., Kim, B. J., Hara, S. A., Lee, C. D., Gutierrez, C. A., Hoang, T. Q., & Meng, E. (2013). Novel flexible Parylene neural probe with 3D sheath structure for enhancing tissue integration. Lab on a Chip, 13(4), 554-561.
13 Kim, J. H., Lee, G. H., Kim, S., Chung, H. W., Lee, J. H., Lee, S. M., Kang, S. Y., & Lee, S. H. (2018). Flexible deep brain neural probe for localized stimulation and detection with metal guide. Biosensors and Bioelectronics.
14 Felix, S. H., Shah, K. G., Tolosa, V. M., Sheth, H. J., Tooker, A. C., Delima, T. L., Jadhav, S. P., Frank, L. M. & Pannu, S. S. (2013). Insertion of flexible neural probes using rigid stiffeners attached with bio dissolvable adhesive. Journal of visualized experiments: JoVE, (79).
15 Zhao, Z., Kim, E., Luo, H., Zhang, J., & Xu, Y. (2017). Flexible deep brain neural probes based on a parylene tube structure. Journal of Micromechanics and Microengineering, 28(1), 015012.
16 Fried, et al, “Cerebral microdialysis combined with single-neuron and electroencephalographic recording in neurosurgical patients.” Journal of neurosurgery 1999, 91 (4), 697-705.
17 Maynard, E. M.; Nordhausen, C. T.; Normann, R. A., The Utah intracortical electrode array: a recording structure for potential brain-computer interfaces. Electroencephalography and clinical neurophysiology 1997, 102 (3), 228-239
18 Steinmetz, N. A. et al, “Neuropixels 2.0 A miniaturized high-density probe for stable, long-term brain recordings.” Science 2021, 372 (6539)
19 Paulk, A. C et al., “Large-scale neural recordings with single neuron resolution using Neuropixels probes in human cortex.” Nature Neuroscience 2022, 25 (2), 252-263.
20 Chung, J. E. et al, “High-density single-unit human cortical recordings using the Neuropixels probe.” Neuron 2022.
21 Chiang, C.-H. et al, “Flexible, high-resolution thin-film electrodes for human and animal neural research.” Journal of neural engineering 2021, 18 (4).
22 Mancilla, A. A. et al, “Sensing Local Field Potentials with a Directional and Scalable Depth Array: DISC,” bioRxiv 2021.
23 Sellers, K. K. et al, “Thin-film microfabrication and intraoperative testing of pECoG and iEEG depth arrays for sense and stimulation,” Journal of Neural Engineering 2021, 18 (4).
24 Pothof, F. et al., “Chronic neural probe for simultaneous recording of single-unit, multi-unit, and local field potential activity from multiple brain sites,” Journal of neural engineering 2016, 13 (4).

### SUMMARY OF THE INVENTION

A preferred embodiment provides a microelectrode probe device that includes multiple layers of biocompatible polymer films formed in an elongate shape. Micro-electrode recording conductive sites and macro-electrode conductive stimulation sites are exposed from the polymer films near a first terminal end of the elongate shape. Insulated electrical traces within the polymer films are connected to the micro-electrode recording conductive sites and macro-electrode conductive stimulation sites. An elongate hollow within the multiple layers of polymer films extends to micro-electrode recording conductive sites and macro-electrode conductive stimulation sites. The elongated hollow being configured to accommodate a removable stainless-steel stylet. The stainless-steel stylet permits implantation with the probe device wrapped around it, and the stylet can be removed.

### BRIEF DESCRIPTION OF THE DRAWINGS

FIG. 1 is a schematic top view of a preferred high channel count stereo-electrode depth probe (D-Probe) device of the invention;

FIGS. 2A-2K are schematic cross-sectional views that show a preferred fabrication method of the FIG. 1 D-Probe device;

FIG. 3 shows a preferred fabrication process flow for the FIGS. 2A-2K fabrication method;

FIGS. 4A-4D are images of an experimental D-Probe device consistent with FIG. 1;

FIG. 5 is a schematic top view of preferred micro Stereoelectroencephalography (μSEEG) device of the invention;

FIGS. 6A-6V are schematic cross-sectional views that show a preferred fabrication method of the FIG. 5 μSEEG device; and

FIG. 7 shows a preferred fabrication process flow for the FIGS. 6A-6V fabrication method.

### DETAILED DESCRIPTION OF THE PREFERRED EMBODIMENTS

Preferred embodiments provide a thin stylet-guided and scalable high-channel count intracranial electroencephalography (iEEG) depth probe (D-probe) for clinical recording and stimulation. The preferred stylet-guided D-probe incorporates a hollow structure near the center of its body to permit the insertion of a stylet that provides stiffness needed for implanting the probe yet retains the flexibility of the device when the stylet is extracted after implantation. A remaining thin polymer layer is compliant to the pulsating brain and displaces a minimal tissue volume. This encourages and permits tissue regeneration next to the D-probe post implantation.

Preferred D-probes provide a hollow structure—similar to current tubular clinical electrodes but in thin polymer layers that can be manufactured with thin-film processing techniques—inside a polymer substrate where a removable stainless-steel stylet resides. With the stable structure, the size of the device is scalable and allows an extended length of probe with a stylet that can guide insertion into deeper region of the human brain, such as the hippocampus while laminar recording from superficial layers can be easily performed.

The D-probe is fabricated using a preferred method of surface micromachining of thin-flexible substrates and incorporate highly electrochemically active electrode sites including poly(3,4ethylenedioxythiophene):poly(styrenesulfonate) (PEDOT:PSS), platinum nanorods, or others, with low contact impedances enabling the high fidelity recording and safe stimulation of brain activity.

D-probes of the invention can be used in electrophysiological investigations and treatment of neurological disorders by electrical stimulation in well-known procedures such as deep brain stimulation (DBS) for a wide population of patients with epilepsy, movement disorders, and others.

Preferred probes of the invention provide a high-channel count depth probe suitable for use in humans. Methods provide for scalable fabrication. A preferred example D-probe is much longer than typical 10 mm long probes, e.g., at least 30 mm, and for example currently 90 mm long, and is preferably composed of tightly spaced (60 microns) microelectrodes with 64 channels for recording or microstimulation of larger macro-electrodes for conventional clinical stimulation. A preferred D-probe utilizes only one metallization layer to achieve the large number of contacts compared to clinical electrodes (<10). Tighter metal line spacing and the utility of multi-metallization layers permits a D-probe of the invention to provide more than 100 channels. Additionally, the length of the D-probe permits implantation of the microelectrodes into deep brain structures to the hippocampus and longer versions of the D-probe can be manufactured using the present scalable microfabrication techniques either on larger substrates (>4-inch) or by curving the metal lines in a snake-like pattern on smaller substrates (4-inch).

An example preferred μSEEG electrode tested in non-human primates is 130 mm long composed of tightly spaced (60 microns) microelectrodes with 128 channels for recording or microstimulation of larger macro-electrodes for conventional clinical stimulation. This number of contacts (channels) in the μSEEG can be increased by adding multiple metallization layers. A single metallization permitted 128 contacts compared to maximum of 16 contacts for clinical electrodes. Tighter metal line spacing and the utility of multi-metallization layers are readily achievable by present manufacture methods to increase the number of channels. The length of the μSEEG permit implantation of the microelectrodes, for example, into deep brain structures to the hippocampus. The example μSEEG electrodes were made on larger substrates 7-inch glass substrates, and panels that can much larger, e.g. 3.1×3.1 m2; therefore, the manufacturing can be scaled to make many copies of the μSEEG in the same run and at a cheap cost.

Laminar probes (length of a few millimeters) can also be made for recording from sacrificial layers of the brain. Additionally, multiple parallel (or multiple finger) D-probes of the invention can be manufactured on the same substrate permitting the 3D recording and stimulation from multiple regions and with wide coverage of the human brain.

Preferred D-probes of the invention are formed from a fabrication process that is compatible with different types of polymers (e.g., ParyleneC and polyimide). The present fabrication process allows selection of design and size of the device based on target of interest for animal, primates and human use, for lengths extending from tens to hundreds of millimeters. The present D-probe is composed of biocompatible polymers (ParyleneC or polyimide) as the substrate for fabrication as well as the body of the final device, biocompatible metal traces (Cr, Pt), conductive layer (PEDOT:PSS, or PtNRs) and medical grade stainless stylet. Other biocompatible materials that can be processed with the present manufacturing process can also be used.

Preferred embodiments provide low impedance for high SNR recording: PEDOT:PSS or PtNRs as conductive layer achieves low impedance (1 kHz impedance <100 kΩ), essential for high SNR recording and efficient and safe stimulation.

Preferred embodiments provide compatibility of the overall manufacturing process with thin-film transistor technology, such as indium gallium zinc oxide (IGZO) transistors for signal conditioning and multiplexing for both recording and stimulation, which enables the multiplication of the channel count in human-compatible depth probes to thousands of contacts.

Preferred stylet guided electrodes can also be used to target other parts of the nervous system. Miniature spinal cord recording and stimulation probes can made by preferred methods. The stylet-guided electrode can permit the insertion of a thin layer of high-channel count microelectrodes above or below the dura (sub=dural) and can bend to insert the electrodes on the ventral side of the spinal cord near motor fibers, enabling highly efficient neuro modulation devices for a variety of movement disorders, chronic pain, and others. Interfacing with the peripheral nervous system and at various locations of the body can be accomplished with the D-probe enabling higher efficacy and safer stimulations to mitigate disorders in the nervous system.

Preferred embodiments provide thin stylet-guided and scalable high-channel count intracranial micro-stereo-electroencephalography (μSEEG) depth probe for clinical recording and stimulation. The μSEEG is developed with a preferred method for thin-film electrodes. The method provides reproducible, customizable, and high throughput production of electrodes (1) to be implanted in the operating room using similar brain implant techniques to standard clinical depth electrodes, and (2) to reach deep brain structures and achieve high spatial resolution and channel count with a much thinner electrode body. A preferred method exploits (1) sacrificial layers employed in the microfabrication of free-standing microelectromechanical systems (MEMS) devices. A stylet inserted where the Ti sacrificial layer is removed assists in hardening and implanting the depth electrode—similar to the standard clinical SEEG electrode implantation procedures—and is subsequently removed. The removal of the stylet leaves a thin polymer layer that is compliant to the pulsating brain and that displaces a minimal tissue volume permitting tissue regeneration next to the μSEEG post implantation. (2) The preferred MEMS process can be implemented on relatively large (e.g., 18×18 cm²) glass substrates, which permits fabrication of multiple copies of the SEEG devices. Thin-film based and clinical-grade depth microelectrode arrays are provided with flexibility in design and scalability afforded by the glass substrate based manufacturing, which is cost effective, and does not involve manual assembly typical for standard SEEG electrodes. The μSEEG dimensions can be selected over a wide range to provide for application-based contact spacing and channel count.

An μSEEG of the invention can be used to target the thalamus, substantia nigra and other subcortical structures for the control of seizures, Parkinson's disease, and essential tremor as well as a growing number of other disorders. Additional applications include diagnosis of memory disorders and assistance in memory restoration. Another application is to a brain-computer interfaces to restore movement and communication in the setting of trauma, amyotrophic lateral sclerosis and stroke. Similar to clinical SEEG electrodes, the μSEEG electrode can be implanted through small openings in the skull and penetrate the brain parenchyma at varying depths depending on the surgical target, and allow for subcortical recordings and, sulcal depth evaluation, with deep structural reach that is not attainable by surface electrodes.

A preferred example μSEEG electrode can be implanted with stylets in place, and after implantation and reaching the target brain structure, the stylets can be retracted so that only the μSEEG remains in the brain while the diagnosis/treatment is conducted for <30 days. This can help to reduce the biofouling around the μSEEG resulting from the different Young's modulus of the brain and the stylet.

A preferred example μSEEG electrode is reconfigurable, monolithically integrated human-grade flexible depth electrode capable of recording from up to 128 channels and able to record at a depth of 10 cm in brain tissue. This thin, stylet-guided depth electrode is capable of recording local field potentials and single unit neuronal activity (action potentials), validated across species. This device represents a major new advance in manufacturing and design approaches which extends the capabilities of a mainstay technology in clinical neurology.

Preferred embodiments of the invention will now be discussed with respect to experiments and drawings. Broader aspects of the invention will be understood by artisans in view of the general knowledge in the art and the description of the experiments that follows.

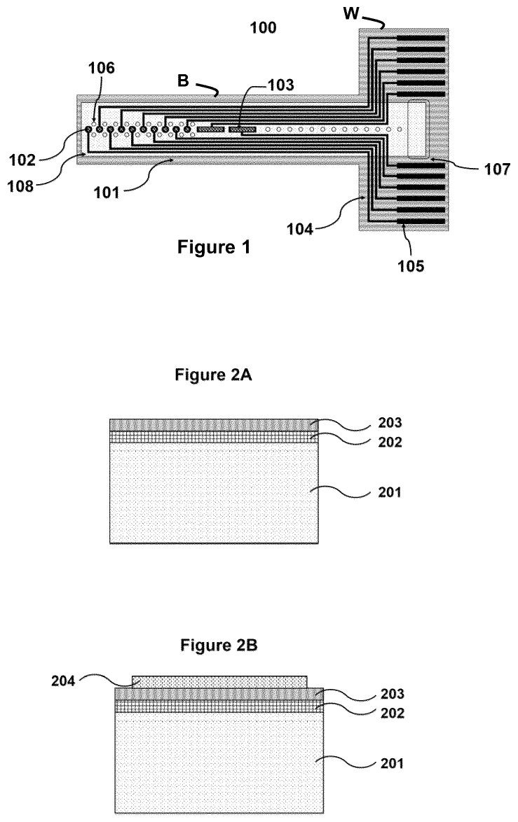

FIG. 1 is a top-view of a preferred D-probe device 100. The device 100 is composed of multiple layers of polymer films 101 formed in an elongate T shape. The polymer films can either be paryleneC or polyimide or any other biocompatible flexible layer that can be processed in thin-film manufacturing techniques. ParyleneC and polyimide are flexible and soft materials with closely matched Young's modulus to the brain which helps minimizing the damage to brain tissue in the semi-chronic and chronic implantations.

The preferred total thickness of the polymer layer is 6˜10 um. A preferred length of the device is ˜90 mm with a width of ˜1.2 mm at the contact region and ˜45 mm at the bonding pad region. Other geometries and dimensions can be easily made through present methods of fabrication for different purposes and for different connectors that will connect electrodes to a clinical montoring system or to a closed loop brain implant. Micro-electrode recording sites 102 and macro-electrode stimulation sites 103 are formed via a coated conductive layer which yields low electrochemical impedance. The sites 102 and 103 are near the narrow end of the elongate T shape. The conductive layer can be PtNRS or PEDOT:PSS or other materials. The diameter and pitch of the conductive layer can vary from sub-micrometer to tens of micrometers based on the electrophysiology requirements.

Cr/Pt metal traces 104 form n channels through the elongate base B of the T shape, e.g. 64 channels in an example experimental D-probe device. FIG. 1 shows fewer channels for simplicity of illustration. Cr/Pt metal traces are expanded to bonding pads 105 at an opposite wide top end W of the T shape, where anisotropic conductive film (ACF) and ribbon cable are bonded for the connection to a recording system or another device. Multi-metallization layers can be used.

A medically grade stainless-steel stylet 108 is inserted through an elongate hollow opening 107. The stylet stays inside the polymer layer 101 during implantation of the D-Probe device 100 and can be extracted after implantation. An example diameter of the stainless-steel stylet is 0.025 mm, and a range of preferred diameters is ˜0.01-0.025 mm. Via etch holes 106 on paryleneC/polyimide provide openings to etch a sacrificial layer to form the elongate hollow opening.

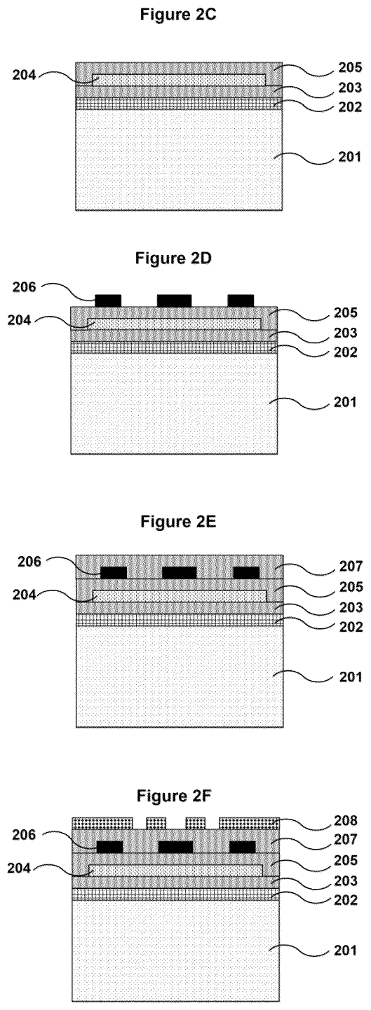

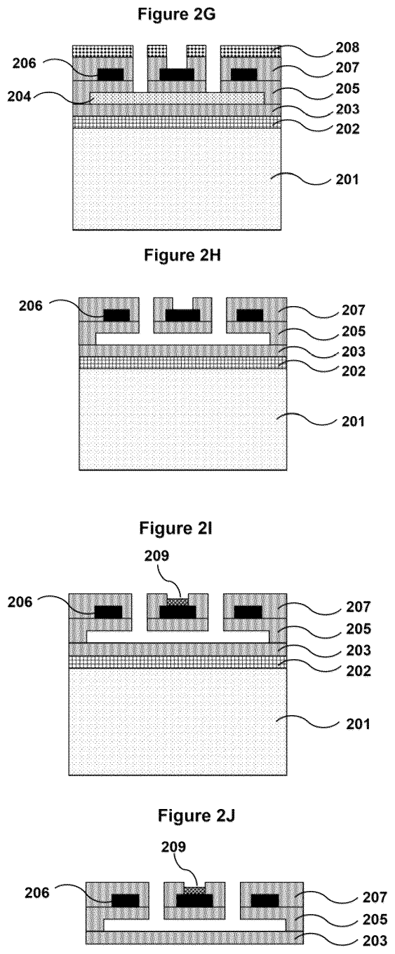

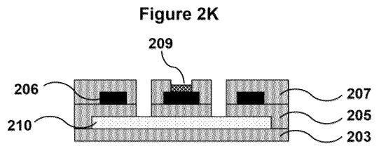

FIGS. 2A-2K show a preferred fabrication method of the FIG. 1 D-Probe device. In FIG. 2A, a polymer layer 203 is deposited on top of an anti-adhesion/sacrificial layer 202 on a carrier substrate 201. Standard 4 inch or larger substrates can be used as the carrier substrate 201, which can be Si or a glass wafer. Polymer layer 203 can be paryleneC or polyimide. A ParyleneC layer is deposited by ParyleneC coater by chemical vapor deposition and the polyimide is spin-casted and cured in an oven. Micro-90 is used as an anti-adhesion layer 203 when paryleneC is used as the substrate/thin film body of the D-probe, and when polyimide is used as the body of the D-probe, an Al layer of 500 nm is utilized as a sacrificial layer 203.

In FIG. 2B, sacrificial layer 204 is patterned by photolithography and deposited by sputtering on polymer layer 203. The sacrificial layer can be composed of 10 nm of Cr as an adhesion layer and 200 nm of Au for elective etching post the manufacture of top layers of the D-probe. The Au sacrificial layer will be later removed to create the hollow structure inside the polymer layers.

A second layer of polymer 205 is deposited in FIG. 2C. The layer 205 and seals the sacrificial/polymer layer 204.

FIG. 2D shows a step of Cr/Pt (10/100 nm) metal trace 206 being formed. This formation can be conducted via photolithograph and sputtering.

FIG. 2E shows that another layer of polymer 207 is deposited. This layer 207 serves to encapsulate the metal trace 206.

FIG. 2 F shows creation of an etch mask 208 by photolithography. The etch mask 208 can be Cr, Al or SU8.

FIG. 2G shows via etching. A via is etched through polymer layer 207 and 205 by oxygen plasma reactive ion etching. The via etching is conducted to open electrode recording sites for electrochemical recording and or stimulation, to open bonding pads for cable bonding, to etch Au sacrificial layer 204, and to create an opening for stylet insertion.

FIG. 2 H shows sacrificial layer etching. After the etch mask 208 is removed, the Au sacrificial layer 204 is etched by chemical wet etching with Au etchant through a via opening.

FIG. 2I shows conductive layer 209 deposition step at the electrode recording sites. PEDOT:PSS is deposited by electrochemical deposition. The electrochemical deposition is preferably conducted to include a three-electrode configuration, electrode recording sites (Pt) as working electrode, Ag/AgCl as the reference electrode and Pt wire as the counter electrode. The deposition can be conducted in a polymerization solution composed of 3,4ethylenedioxythiophene (EDOT) and poly(sodium-4-styrenesulfonate).

FIG. 2J shows release. The boundaries of the D-probe in the polymer layer are cut by a CO₂ laser and are released from the carrier substrate 201 by removing anti-adhesion/sacrificial layer 202. Micro-90 anti-adhesion layer for paryleneC is dissolved by deionized water and Al sacrificial layer for polyimide is removed by anodic dissolution. Anodic dissolution of Al layer can be done inside NaCl solution where the substrate is connected to an external positive terminal of voltage source and Pt electrode is connected to the negative terminal.

FIG. 2 K shows the final structure of the device, created when the stainless-steel stylet 210 is inserted through the via opening. The stainless-steel stylet 210 is slowly inserted under observation from microscope.

The same method can be used repeatedly to produce the insulated electrical traces within the polymer films. This produces a slightly thicker device for each additional layer used for traces, but having the traces in different layers can limit cross-talk in the signals from the traces, which can occur if the number of channels is increased while the distance between traces in the same layer is reduced.

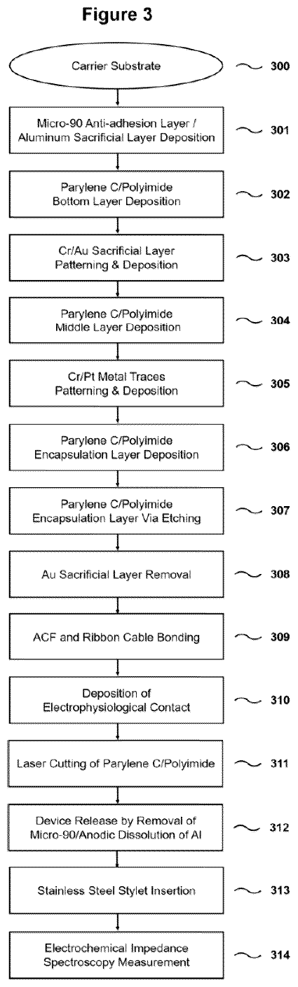

FIG. 3 shows a preferred fabrication process flow for the FIGS. 2A-2K fabrication method. The fabrication starts with deposition of an anti-adhesion layer 301 on a 4 inch Si substrate 300. ParyleneC/Polyimide bottom layer deposition 302 is followed and Cr/Au sacrificial layer is defined 303 on top of the bottom polymer layer. After depositing paryleneC/polyimide middle layer 304, definition of Cr/Pt metal trace 305 is followed. The final paryleneC/polyimide layer is deposited to encapsulate metal traces 306 and electrode sites for recording and openings for sacrificial layer wet etching are via etched 307. Au sacrificial layer is removed 308 to make hollow structure for stylet insertion and ACF/ribbon cable bonding is followed 309 for connection to the recording system. On the via etched electrode recording sites, conductive layer such as PEDOT:PSS or PtNRs is deposited 310 and the contour of the device is cut by a CO₂ laser. The paryleneC/polyimide device is released by dissolving micro-90 layer for the former and anodic dissolution of Al sacrificial layer. A stainless-steel stylet is inserted through the via etched opening 313 and the electrochemical impedance spectroscopy of the final device is measured in a phosphate buffered saline solution with Ag/AgCl reference electrode and Pt wire counter electrode 314 to test the electrode prior to deploying for sterilization and clinical use.

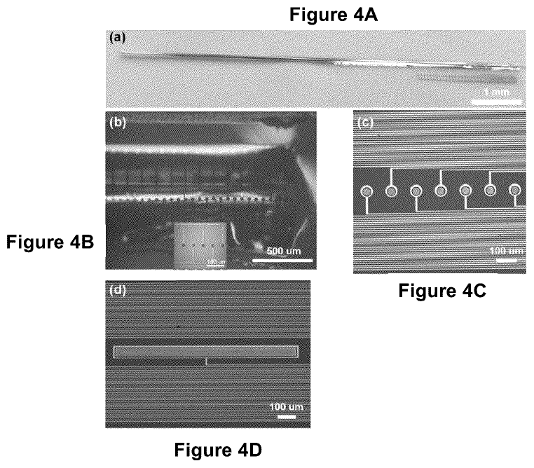

FIG. 4A is a perspective image of an experimental device with the stylet inserted. In FIG. 4A, the multiple layers of polymer films are wrapped around the removable stainless-steel stylet in a configuration for implantation. In this configuration, the device has an example approximate total with of 1.2 mm. FIG. 4B is an optical microscope image at the tip of the device with the stylet inserted. FIG. 4C is an optical microscope image of the microelectrode (sensing) sites, and FIG. 4D of a macro-electrode (stimulation) site.

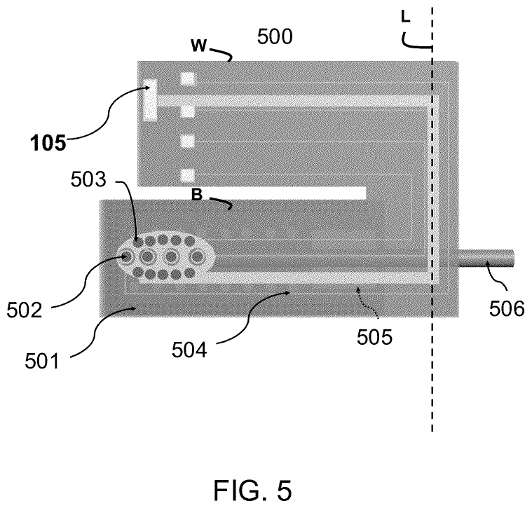

FIG. 5 is a top view of a preferred μSEEG device 500. The device 500 includes multiple elongate layers of polymer films 501 formed into a U Shape with an elongate narrow leg B and an elongate wider leg W. The polymer films can either be paryleneC or polyimide or any other biocompatible flexible layer that can be processed in thin-film manufacturing techniques. ParyleneC and polyimide are flexible and soft materials which when made thin, they can closely matched Young's modulus to the brain tissue which helps minimizing the damage to brain tissue in the semi-chronic and chronic implantations. The total thickness of the polymer layer is preferably 10˜15 μm. The length of the recording area of an example preferred device is ˜48 mm with a width of ˜1.2 mm and a length of the connector area of the device is ˜280 mm with a width of ˜10 mm. Other geometries and dimensions can be fabricated by present methods for different purposes and for different connectors that will hook the electrodes to the clinical monitoring system or to the closed loop brain implant. The U-shaped device 500 is useful, for example, to make the total device length longer than a substrate size used to form the device. As an example, with a seven-inch substrate, the device shape during fabrication on the substrate can be a U-shape. After release from the substrate, one of the narrow leg B and wider leg W can be flipped (rotated by 180 degrees about an axis L of the material between the two legs) to create a device that is twice the length of the substrate, e.g. for a seven-inch substrate the flip produces a ˜14 inch long device.

The micro-electrode recording sites 502 and macro-electrode stimulation sites 503 are coated with a conductive layer which yields low electrochemical impedance. The conductive layer can be PtNRs or PEDOT:PSS or other materials. The diameter and pitch of the conductive layer can vary from sub-micrometer to tens of micrometers based on the electrophysiology requirements. Cr/Au metal traces 504 form 128 channels for micro-electrodes and 64 channels for macro-electrodes in the proof-of-concept μSEEG device. Only a few channels are shown in FIG. 5 for simplicity of illustration. Cr/Au metal traces are expanded to bonding pads 505 where a printed circuit board (PCB) is bonded for the connection to the recording system. Other connectors can be used including anisotropic conductive film (ACF) tape and ribbon cable. Multi-metallization layers can be used. A medically graded stainless-steel stylet 506 is inserted through via opening 507 in the elongate narrow leg B and stays inside the polymer layer 501 during implantation and can be extracted after implantation. An example diameter of the stainless-steel stylet is 0.25 mm.

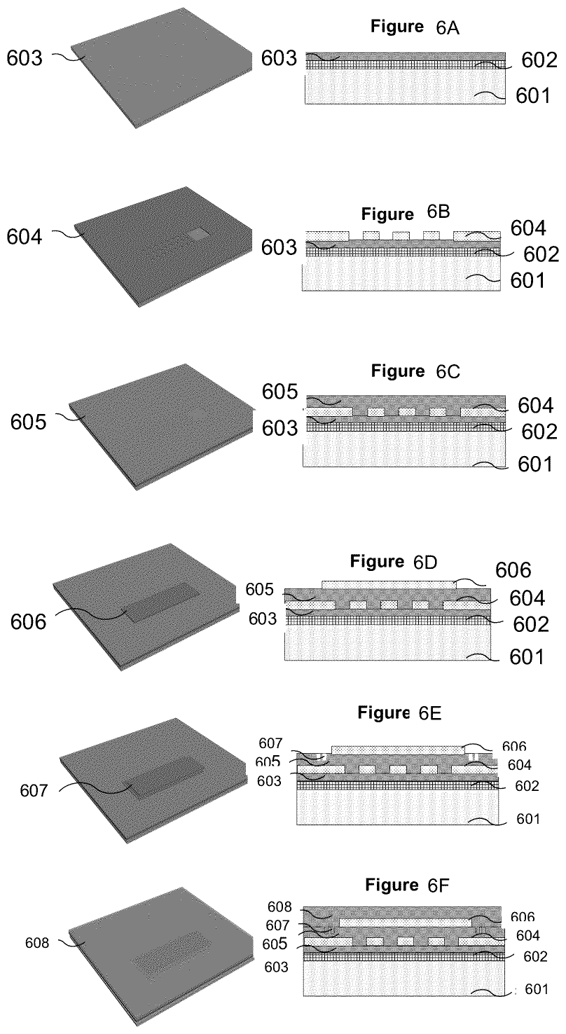

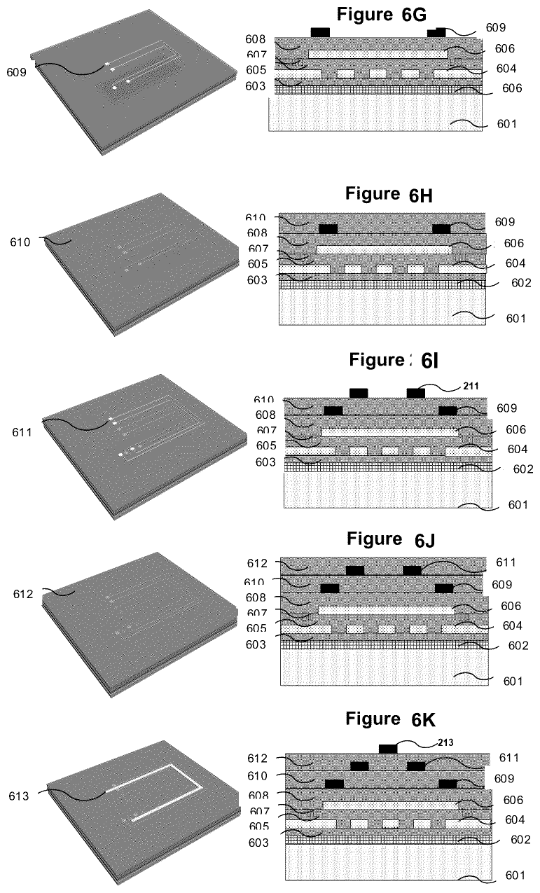

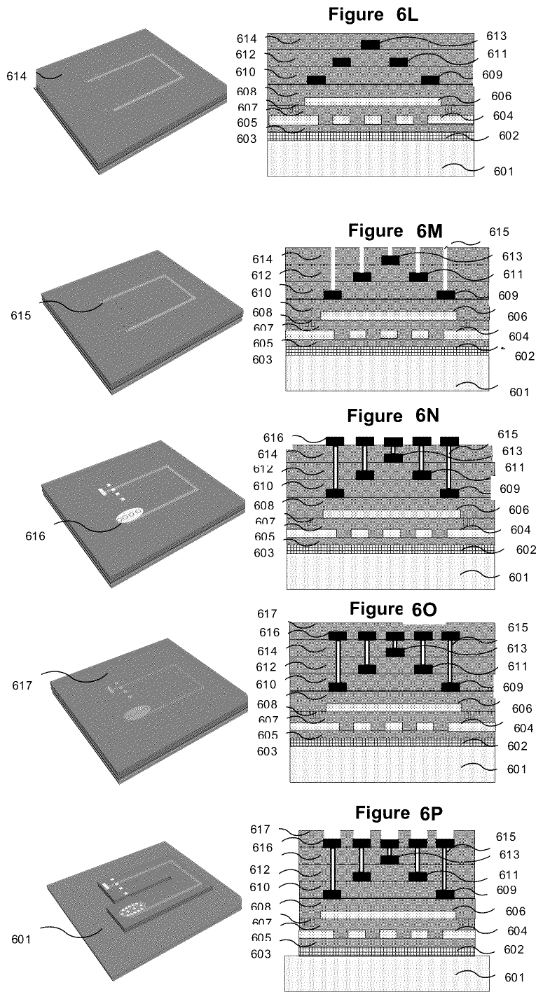

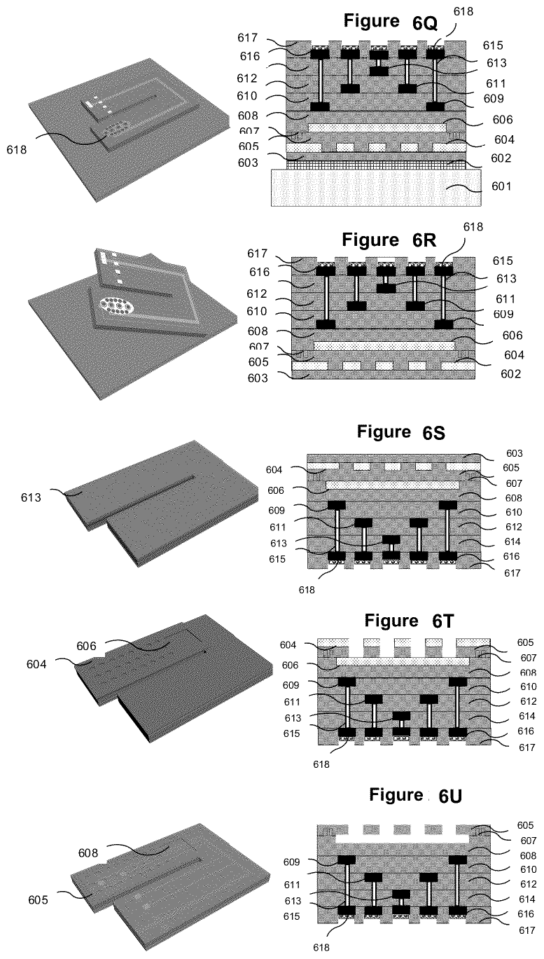

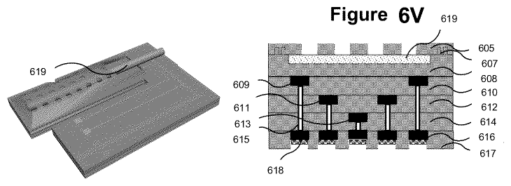

FIGS. 6A-6V show a preferred fabrication method of the FIG. 5 μSEEG device In FIG. 6A, a sacrificial polymer layer 603 is deposited on top of anti-adhesion/sacrificial layer 602 on a carrier substrate 601. In experiments, a 7-inch wide substrate was used, but other sizes can also be used. Carrier substrate 601 can be Si or glass or other wafer that is compatible with thin film manufacturing.

The polymer layer 603 can be paryleneC or polyimide or other thin-film biocompatible polymer. ParyleneC layer is preferably deposited by chemical vapor deposition and the polyimide is spun-casted and then cured in an oven. Micro-90 is used as an anti-adhesion layer 602 when either paryleneC or polyimide is used as the substrate/thin film body of the μSEEG.

In FIG. 6B, a Ti mask layer 604 is patterned by photolithography and deposited by sputtering on polymer layer 603 for net structure formation on the back of the μSEEG in later processes. The Ti mask layer for net structure is composed of 40 nm of Ti. A 1st layer of polymer 605 is deposited and seals the Ti mask layer/polymer surface as shown in FIG. 6 C. FIG. 6D shows another 40-nm-thick Ti layer to be used as a sacrificial layer 606, which will be later removed to create hollow structure inside the polymer layers. Microhole arrays 607 are patterned along the sacrificial layer 606 to increase the surface area of the 1st polymer layer 605 so that adhesion of the 1st polymer layer 605 and the 2nd polymer layer 608 can be enhanced (FIG. 6E). The 2nd polymer layer 608 is spun cast and seals the Ti sacrificial layer 606 and microhole arrays 607, as shown in FIG. 6F.

The fabrication process with multi-layer metallization was used to create an experimental device for 128 microelectrodes for recording contacts and 16 macro-electrodes for stimulation contacts. The dimensions and thicknesses given are consistent with the experimental device but do not limit the method generally. Metallization layers can be added or subtracted from the fabrication process to yield the desired number of contacts.

Photolithography is used to define the traces, and sputtering can used to deposit Cr/Au (10/250 nm) metal trace lines for the micro-electrodes leading to structures 609 on the very top surface of the layers as illustrated in FIG. 6G. Then, another layer of polymer (a 3rd polymer layer) 610 is deposited to encapsulate the metal trace 609 as shown in FIG. 6H. Another Cr/Au (10/250 nm) metal trace for 64 micro-electrodes 611 is formed using the same method as shown in FIG. 6I, and another layer of polymer (a 4th polymer layer) 612 is deposited to encapsulate the metal trace 611 as shown in FIG. 6J. Lastly, a third Cr/Au (10/500 nm) metal trace for 16 macro-electrodes 613 is patterned (FIG. 6K), and passivated by another layer of polymer (a 5th polymer layer) 614 as shown in FIG. 6L. Then, polymer layers including 610, 612, and 614 are etched to form via holes 615 (FIG. 6M) to expose the Cr/Au metal traces including 609, 611, and 613. Via holes 615 are formed by photolithography and dry-etching techniques, usually with an oxygen plasma. Subsequently, Cr/Au (10/500 nm) pads 616 are deposited using non-directional sputtering on the via opened region 615 to connect with metal traces including 609, 611, and 613. Non-directional sputtering is used to cover the sidewalls of the via and enable conductivity from exposed regions of metal traces 609, 611, and 613 to pads 616 (FIG. 6N). An encapsulation layer of polymer (a 6th polymer layer) 617 is then deposited as shown in FIG. 6O and via holes are opened by via-etching process including photolithography and dry-etching techniques to expose pads 616 as shown in FIG. 6P. During this step, outline of the μSEEG is also patterned and etched. PEDOT:PSS or PtNRs 618 are deposited onto the via-opened pads 615 (FIG. 6Q). PEDOT:PSS is formed by electrochemical deposition. The electrochemical deposition is conducted by three-electrode configuration, electrode recording sites (Pt) as working electrode, Ag/AgCl as the reference electrode and Pt wire as the counter electrode in a polymerization solution composed of 3,4-ethylenedioxythiophene (EDOT) and poly(sodium-4-styrenesulfonate). PtNRs are formed by deposition of ˜0.5 μm thick PtAg alloy using a co-sputtering technique performed at 400 W (RF) and 35 W (DC) powers for co-deposition of Ag and Pt, followed by dealloying of Ag in nitric acid at 60° C. for 2 min.

Whole layers are immersed into deionized water (DI water) to dissolve anti-adhesive layer 602 and device layers are separated from the carrier wafer 601 (FIG. 6R). After releasing the μSEEG structures, another via etching is performed from back side of the structures using oxygen plasma in a reactive ion etcher to remove the sacrificial polymer layer 603 and form net structures in exposed holes of the 1st polymer layer 605 using oxygen plasma reactive ion etching through the Ti mask layer for net structure 604 formation as shown in FIG. 6S and FIG. 6T. The Ti mask layer 604 and Ti sacrificial layer 606 are etched by chemical wet etching with buffered oxide etch through via opening as depicted in FIG. 6U. FIG. 6V shows the final structure of the device where stainless-steel stylet 619 is inserted through a sheath between polymer layers including 605 and 608. The stainless-steel stylet 619 is slowly inserted under observation from microscope.

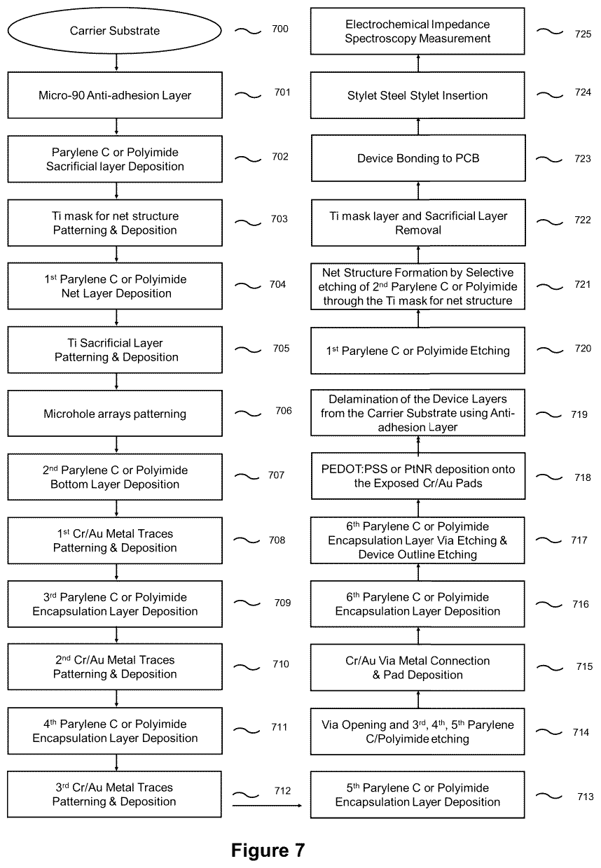

FIG. 7 shows a preferred fabrication process flow, which starts with deposition of an anti-adhesion layer 701 on a 7 inch glass substrate 700. ParyleneC or polyimide sacrificial layer deposition 702 is followed and Ti mask layer for net structure is defined 703 on top of the sacrificial polymer layer. After depositing a 1st paryleneC or polyimide net layer 704, Ti sacrificial layer is formed using photolithography and metal deposition technique 705. Subsequently, microhole arrays are patterned along the Ti sacrificial layer to enhance adhesion between 1st and 2nd paryleneC or polyimide layer 706. Then, a 2nd paryleneC or polyimide layer is deposited 707 and definition of 1st Cr/Au metal trace for 64 micro-electrodes is followed 708. A 3rd paryleneC or polyimide encapsulation is deposited 709 and 2nd Cr/Au metal trace for another 64 micro-electrodes is deposited 710. Lastly, a 4th paryleneC or polyimide encapsulation layer is deposited 711 and 3rd Cr/Au metal trace for 16 macro-electrodes is deposited 712. 5th paryleneC or polyimide encapsulation layer is deposited 713 and via holes are defined by photolithography and 3rd, 4th, and 5th paryleneC or polyimide encapsulation layers are dry etched in exposed regions 714. Subsequently, Cr/Au pads were deposited using sputtering method on the via opened region connected with 1st, 2nd, and 3rd metal traces 715. The entire surface is then encapsulated by 6th paryleneC or polyimide layer 716. Then, 6th paryleneC or polyimide is via opened by via-etching process while outline of the μSEEG is etched using photolithography and dry-etching techniques 717. PEDOT:PSS or PtNR is deposited onto the exposed Cr/Au pads 718. The paryleneC/polyimide device is released by dissolving micro-90 layer 719. Then, 1st paryleneC or polyimide layer is etched using oxygen plasma reactive ion etching 720. Net structure is formed by via etching using oxygen plasma reactive ion etching through the Ti mask layer for net structure 721. Ti mask layer and sacrificial layer are removed 722 to make hollow structure for stylet insertion. Then, μSEEG is bonded with PCB for connection to the recording system 723. A stainless-steel stylet is inserted through the via etched opening 724 and the electrochemical impedance spectroscopy of the final device is measured in a phosphate buffered saline solution with Ag/AgCl reference electrode and Pt wire counter electrode 725 to test the electrode prior to packaging to deploy for sterilization and clinical use.

Microelectrode probe devices of the invention, as exemplified by the devices 100 and 500 above can be tailored in its range of depth to sample cortical and or deep structures in the brain (or both). Testing of 64-channel and 128-channel devices consistent with the device 100 of FIG. 1 demonstrated stimulation of neural activity and recording of clinically relevant neural dynamics.

A 128-channel long μ SEEG electrode was built according to FIG. 1 to most resemble clinical depth electrodes with a working recording length of 7.65 mm at the tip of the electrode that is, overall, 28 cm long, 1.2 mm wide, and 15 μm thick which would allow insertion and recording from deeper brain structures. The electrode contacts in this design are concentrated at the tip of the device with inter-contact distances of 60 μm. To test if we could record single neuron activity at depth, we recorded neural dynamics in an nonhuman primate that was awake but resting and viewing flickering light-emitting diodes (LEDs) to test for visual responses. The long μ SEEG electrode was held by a microelectrode microdrive to drive the microelectrode to multiple depths from the surface of the cortex within an implanted chamber. Along the trajectory moving toward the thalamus, we stopped and recorded at three different depths to examine spiking activity in the cortex as well as in white matter. At depths 1 and 2, we found we could record spikes which clustered into single-unit and multi-unit activity (MUA) (depth 1:1 MUA cluster, 4 single-unit clusters; depth 2:5 MUA clusters, 31 single-unit clusters), which we determined by examining the autocorrelation of the spike times and the waveform consistently through time using Kilosort. No identifiable single-unit activity at was found at depth 3, as the microelectrode was likely mostly in white matter at that depth. The units and MUA clusters were distributed at different distances and locations along the 128 contacts of the long μ SEEG with a range of spike rates, most of which were around 2 Hz. The waveform measures show that the units sampled at depths 1 and 2 were clustered in amplitude, the peak-trough ratio, and spike duration measures compared to the MUA clusters found at depth 3. In other words, these clusters are more likely single-unit activity or putative neurons since they were detected while the recording contacts were in cortex but not while in white matter.

The fabrication methods described above are readily scalable, and the number of contacts can be increased well beyond 128 channels in experimental microelectrodes, and at a much lower manufacturing cost than prior clinical and other research depth electrodes. A 240 sq. in. monitor has a retail price of nearly $100 with active transistors and light-emitting diodes. The same area can be used to manufacture at least 20μ SEEG electrodes, pointing to significantly lower costs than current clinical SEEG electrodes (>1000 per electrode) when manufactured at scale. This cheaper advanced manufacturing approach and the added spatial resolution and sensitivity to cellular activity in a smaller form factor is expected to advance the ability to study and treat the human brain and to broaden the access of the technology to underserved communities and other brain diseases.

### Example Fabrication Process to Produce Experimental Microelectrode Probe Devices.

Polished and cleaned soda lime glass plates were used as substrates. A release layer of Micro-90 diluted with deionized water was spin-coated onto the glass. A sacrificial 5-μm-thick polyimide layer (PI—2611, HD MicroSystems) was then deposited. This layer would later separate the device layers from the glass plate. A Ti hard mask was formed for net layer formation, followed by standard lithography, descum, metal deposition, and lift-off processes. The Ti hard mask contained via patterns for hole arrays and a rectangular shape for a sheath. Next, another polyimide layer was applied, serving as the sacrificial bottom layer with holes. A Ti sacrificial layer was deposited to act as an etchstop layer. Adhesion between the layers was increased by patterning hole arrays. The 1st PI layer was then selectively etched, and the photoresist layer was removed. Afterward, the glass substrate underwent baking, and a 2nd PI layer was applied. Metal traces were formed on the 2nd PI layer, composed of Cr/Au(10/250 nm). This process was repeated to create double-layered metal leads. A PtAg alloy was selectively formed on the micro-contact recording sites. A Ti capping layer was added to prevent oxidation. A 3rd polyimide layer was applied, followed by a Ti hard mask. Etching processes exposed the Ti layer deposited during Ti hard mask for net layer formation on the sacrificial PI layer. Another photolithography and etching process was applied to open via holes for recording sites and contact pads. The exposed PI layers were etched, and a Ti passivation layer and paryleneC were deposited to protect PI layers against dealloying. The Ti passivation layer and paryleneC layers were then patterned and etched selectively on the recording site regions to expose the PtAg alloys. Dealloying of PtAg alloys was performed. Then, the paryleneC and Ti passivation layers were removed. The electrodes were delaminated from the glass substrate. The delaminated electrode was transferred onto another carrier glass wafer. Hole arrays were formed on the 1st PI layer by etching through the sacrificial PI layer and the Ti hard mask for net layer formation. The Ti hard mask for net layer formation and Ti sacrificial layers were dissolved in BOE, and the electrode was rinsed with DI water.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A microelectrode probe device, comprising:
multiple layers of biocompatible polymer films formed in an elongate shape;
micro-electrode recording conductive sites and macro-electrode conductive stimulation sites exposed from the polymer films near a first terminal end of the elongate shape;
insulated electrical traces within the polymer films connected to the micro-electrode recording conductive sites and macro-electrode conductive stimulation sites; and
an elongate hollow within the multiple layers of polymer films extending to micro-electrode recording conductive sites and macro-electrode conductive stimulation sites, the elongated hollow being configured to accommodate a removable stainless-steel stylet.

2. The microelectrode probe device of claim 1, comprising at least one removable stainless-steel stylet in the elongated hollow.

3. The microelectrode probe device of claim 2, wherein the at least one removable stainless-steel stylet is at least ˜30 mm long.

4. The microelectrode probe device of claim 3, wherein the at least one removable stainless-steel stylet is at least ˜90 nm long.

5. The microelectrode probe device of claim 3, comprising 64 or more of the micro-electrode recording conductive sites connected to individual corresponding ones of the insulated electrical traces.

6. The microelectrode probe device of claim 3, comprising 128 or more of the micro-electrode recording conductive sites connected to individual corresponding ones of the insulated electrical traces.

7. The microelectrode probe device of claim 2, wherein at least a portion of the multiple layers of polymer films including the micro-electrode recording conductive sites and macro-electrode conductive stimulation sites is wrapped around the removable stainless-steel stylet in a configuration for implantation.

8. The microelectrode probe device of claim 2, comprising via connections between layers and multiple traces in different layers connected to the micro-electrode recording conductive sites and macro-electrode conductive stimulation sites.

9. The microelectrode probe device of claim 2, comprising bonding pads at an opposite end of the micro-electrode recording conductive sites.

10. The microelectrode probe device of claim 2, comprising bonding pads at an opposite end of the micro-electrode recording conductive sites.

11. The microelectrode probe device of claim 10, wherein the elongate shape is a T shape, and the opposite end comprises a wide top end of the T shape.

12. The microelectrode probe device of claim 10, wherein the elongate shape is a U shape with a narrow elongate leg and a wider elongate leg, and the opposite end comprises the wider elongate leg.

13. The microelectrode probe device of claim 10, wherein the elongate hollow is in the narrow elongate leg.

14. The microelectrode probe device of claim 1, wherein the stylet comprises a diameter of ˜0.01-0.025 mm.

15. The microelectrode probe device of claim 1, wherein the biocompatible polymer layers comprise ParyleneC or polyimide.

16. The microelectrode probe device of claim 1, wherein the biocompatible polymer layers comprise a total thickness of ˜10 to ˜15 μm.

17. The microelectrode probe device of claim 1, wherein the insulated electrical traces comprise multi-metallization layers.

18. The microelectrode probe device of claim 1, wherein multi-metallization layers comprise Cr/Pt.

19. The microelectrode probe device of claim 1, wherein multi-metallization layers comprise Cr/Au.
